(19) United States
(12) Patent Application Publication
(10) Pub. No.: US 2009/0018776 A1
(43) Pub. Date: Jan. 15, 2009

(54) SYSTEM AND METHOD FOR NORMALIZING DATA IN NUCLEIC ACID AMPLIFICATION PROCEDURES

(76) Inventor: Roger H. TAYLOR, San Diego, CA (US)

Correspondence Address: AGILENT TECHOLOGIES INC, P.O BOX 7599, BLDG E , LEGAL, LOVELAND, CO, 80537-0599, US

(21) Appl. No.: 11/775,240

(22) Filed: Jul. 10, 2007

(51) Int. Cl. G01N 33/48

(52) U.S. Cl. 702/19

(57) ABSTRACT

The present invention provides systems and methods for normalizing data in nucleic acid amplification procedures. In general, the methods include: obtaining input from a user regarding a number of normalizer measurements to use in calculations; obtaining raw fluorescence measurements of one or more probe(s) over multiple cycles of the procedure; obtaining normalizer measurement(s); and normalizing the probe data using the normalizer measurement(s) specified by the user. The present invention also discloses software and a computer readable medium for carrying method steps of the invention. Reduced cycle times, decreased variability, and/or lower Ct values are obtained as a result of the normalization.

### BACKGROUND OF THE INVENTION

1. Field of the Invention

The present invention generally relates to molecular biology and nucleic acid amplification. More specifically, the invention relates to normalization of data in nucleic acid amplification procedures, such as polymerase chain reaction (PCR).

2. Description of Related Art

Polymerase chain reaction (PCR) is known in the art for amplifying nucleic acid target sequences and is used for a wide variety of applications, including: gene expression quantification, expression profiling, genetically modified organism testing, pathogen detection, viral diagnosis, detection of hereditary diseases, and more. Common components involved in PCR procedures include, among other things, a template nucleic acid sequence to be amplified, one or more primers, and a polymerase for creating synthetic copies of the template. PCR experiments typically comprise many cycles, where each cycle consists of three temperature-specific phases: denaturation (or “separation”) of the template, annealing of the primers to the template, and extension of the primer using the polymerase to insert nucleotides into the growing nucleic acid chain using the template as a guide for nucleotide insertion. Examples of PCR instrumentation used in the art are provided in U.S. Pat. No. 6,657,169 and U.S. Pat. No. 7,005,617, for example.

Quantitative PCR (QPCR) is commonly used to quantitatively measure the amount of DNA, cDNA, or RNA in a sample. For example, probe-based QPCR can use fluorescently labeled probes whose fluorescence changes as a result of annealing to a target sequence. The fluorescence signal intensity increases each subsequent cycle in proportion to the amplified target. As a result, the concentration (i.e., quantity) of the target produced in each cycle can be directly correlated to the increase in fluorescence intensity.

Various plots are often used for analysis of PCR data, including amplification plots and standard curves. Data comprising an amplification plot typically result from multiple sources, including a background component (e.g., due to ambient conditions) that has a linear dependence on the cycle number and a nonlinear component (e.g., the amplified signal). The Ct, or threshold cycle, value is the fractional cycle at which the fluorescence signal rises above a specified signal level (the threshold). For samples having the same starting concentration, a smaller Ct value typically corresponds to an assay with increased target sensitivity while a larger Ct value typically corresponds to decreased sensitivity. For different samples within the same assay, a smaller Ct value typically corresponds to a sample with a larger starting concentration. The Ct value is useful in analyzing PCR experimental results because of its relationship to the starting concentration of the target.

The PCR reaction, in general, is highly sensitive to various experimental factors such as cross-contamination, sample carryover, and sample-to-sample variability, as well as other internal or external events. For example, when contamination occurs, non-targeted portions of DNA may be inadvertently amplified, thereby reducing sensitivity to the targeted portions. Various techniques are currently used to minimize the risk of cross-contamination including closed reaction tubes, such as those disclosed in U.S. Pat. No. 6,730,883. In addition, sample-to-sample variability may occur as a result of, for example, pipetting variability, or differences in reflection, transmission, and gain variability.

Using normalization can compensate for signal-level differences between samples and reduce variations due to system factors. Normalization generally refers to dividing the fluorescence intensity of a probe by that of a known reference (normalizer). The measured signal from a normalizer is affected primarily linearly, if at all, by the PCR process such that normalizers provide relatively consistent signals throughout the reaction. Most normalization techniques perform calculations based on normalizer measurements obtained each cycle. These techniques are referred to herein as “point-by-point” normalization techniques. However, because normalizer measurements and calculations are typically performed each cycle, point-by-point normalization techniques may result in longer cycle times. In addition, point-by-point normalization can add inaccuracy and imprecision to a calculation if the normalizer signal does not have a linear dependence on the cycle number.

Overall processing times for QPCR experiments currently take on the order of 90 minutes or longer. Such processing times may not be practical when large numbers of samples need to be analyzed. Moreover, as QPCR is widely used for more applications, additional experiments need to be run on existing machines, resulting in longer wait times.

It is therefore desirable to reduce the time it takes to conduct PCR-based experiments. To this end, various approaches for performing “fast QPCR” have been proposed. One such technique is to use special master mixes including, e.g., DNA polymerases designed for high speed amplification as disclosed, for example, in U.S. Pat. No. 6,528,254 and U.S. Pat. No. 6,548,250. Other methods for reducing QPCR experiment times include the use of fast ramping thermal cyclers. These and other approaches focus on saving time by modifying thermal cycling conditions. Some of these and other applications further include modifying PCR protocols to decrease run times. While these approaches are helpful for reducing cycle times, questions remain as to whether faster QPCR protocols may exhibit decreased levels of sensitivity as well as increased variability (Hilscher et al., “Faster quantitative real-time PCR protocols may lose sensitivity and show increased variability” Nucleic Acids Research Vol. 33, No. 21, e182, 2005).

There remains a need in the art for performing efficient normalization of data in PCR-type experiments while providing sufficient sensitivity, accuracy, and precision (e.g., as exhibited by improved Ct values). Moreover, there exists a need for a PCR normalization algorithm implemented in software that provides quicker results, provides sufficient sensitivity, accuracy, and precision, and is easy to use. There further remains an ongoing need to reduce the run-times of PCR-based experiments (in addition to, or separate from, existing prior art techniques), thereby increasing the amount of runs that can be performed, maximizing throughput, and reducing experimentation costs.

### SUMMARY OF THE INVENTION

The present invention addresses needs in the art by providing solutions to one or more of the above described drawbacks. The present invention provides systems, methods, and software for accurate nucleic acid amplification that can be performed more efficiently and with improved sensitivity as compared to systems available currently in the art.

According to one aspect, the invention provides a method for normalizing nucleic acid amplification data. In general, the method comprises: obtaining raw fluorescence intensity measurements of one or more probes over two or more (i.e., multiple) cycles; and obtaining a number of normalizer intensity measurements. According to the method, the probe data is normalized with respect to the obtained normalizer measurement(s). In some embodiments, the method is practiced in the context of a computer. In such embodiments, the normalized data may be output to a display. In addition, user input may be received from a user regarding the number of normalizer measurements to be obtained. In preferred embodiments, only one measurement of the normalizer in a sample is used to normalize all the probe measurements from the sample. In embodiments, the minimum number of measurements includes the average of two or more measurements. According to further embodiments, a constant value is subtracted from the normalizer measurement and/or probe measurements. Preferably, reduced cycle times, decreased variability and/or lower Ct values for one or more probe are obtained as a result of the normalization.

According to another aspect, the present invention provides a system for normalizing data in nucleic acid amplification procedures. In general, the system comprises: means for obtaining raw intensity measurement data of one or more probes over multiple cycles; means for obtaining a number of normalizer intensity measurement(s); and means for normalizing the probe measurements with respect to the normalizer measurement(s). Additionally, a user input means and display means may also be provided. In preferred embodiments, the means for normalizing is configured to use only one normalizer measurement of a sample to normalize all the probe measurements from the sample. In embodiments, the means for normalizing is configured to use the average of two or more normalizer measurements. According to further embodiments, the normalizing means is configured to subtract a constant value from the normalizer measurement and/or probe measurements. Preferably, the means for normalizing reduces cycle times, reduces Ct values and/or decreases variability for one or more probe as a result of normalization.

According to yet another aspect, the present invention provides a computer program for performing normalization of signals in nucleic acid amplification procedures. In general, the program comprises: receiving input from a user regarding which normalizer measurements to use during the procedure; obtaining raw intensity measurement data of one or more signal generating substances (e.g., probes) over multiple cycles; obtaining normalizer intensity measurements; and normalizing the intensity measurements with respect to the normalizer measurements. Additionally, the computer program may include instructions for sending the normalized probe data to a display. In preferred embodiments, only one normalizer measurement from a sample is used to normalize all the probe measurements from the sample. In embodiments, the average of two or more measurements from a sample is used to normalize the probe measurements from the sample. According to further embodiments, a constant value is subtracted from the normalizer measurement and/or probe measurements. Preferably, lower Ct values, reduced cycle time, and/or reduced variability are obtained for one or more probe as a result of the normalization.

According to yet a further aspect, the present invention provides a computer program product residing on a computer readable medium. In general, the program comprises instructions for performing normalization of data in nucleic acid amplification procedures. The instructions can comprise: obtaining raw intensity measurement data of one or more probes over multiple cycles; obtaining a number of normalizer intensity measurements; normalizing the probe measurements with respect to the normalizer measurements; and outputting the normalized probe measurements to a display. In embodiments, only one measurement of the normalizer from a sample is used to normalize all the probe measurements from the sample. In embodiments, the number of measurements includes the average of two or more measurements. According to further embodiments, a constant value is subtracted from the normalizer measurement and/or probe measurements. Preferably, lower Ct values, reduced cycle times, and/or reduced variability are obtained as a result of the normalization.

### BRIEF DESCRIPTION OF THE DRAWINGS

FIG. 1 depicts an exemplary PCR system of the present invention.

FIG. 2a illustrates an exemplary raw amplification plot of PCR probe and reference fluorescence data.

FIG. 2b illustrates an exemplary baseline subtracted (dR) amplification plot.

FIG. 2c illustrates an exemplary normalized, baseline subtracted (dRn) PCR amplification plot.

FIG. 3a depicts exemplary method steps according to the present invention.

FIG. 3b depicts an exemplary user-input selection means.

FIGS. 4a-d illustrate comparisons of dR and dRn data plots according to embodiments of the present invention.

FIGS. 5a and 5b illustrate spreads of Ct values for different algorithms using principles of the present invention.

### DETAILED DESCRIPTION OF VARIOUS EMBODIMENTS OF THE INVENTION

Reference will now be made in detail to various exemplary embodiments of the invention. The following detailed description is provided to better illustrate certain details of aspects of the invention, and should not be interpreted as a limitation on the scope or content of the invention. For example, the following discussion will address QPCR by way of example; however, it is understood that the invention is not limited to QPCR. Particular applications of the principles disclosed herein may be effectively made to data from other nucleic acid amplification techniques including, but not necessarily limited to, PCR, real time PCR, reverse transcription PCR (RTPCR), and quantitative reverse transcription PCR (QRTPCR). In addition, the term “probe measurements” is used loosely herein, and it should be understood that probe measurements may be related to target measurements and may also refer to other detectable signals, such as optical signals or electrical signals, of interest as compared to a reference or normalizer.

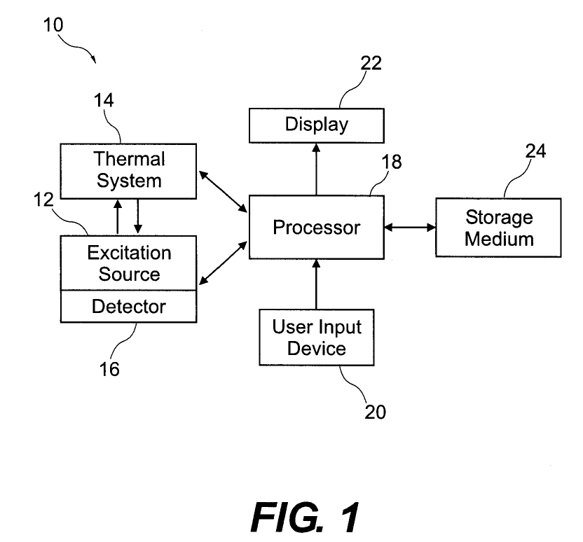

FIG. 1 depicts an exemplary QPCR system 10 according to the present invention. As illustrated in FIG. 1, the QPCR system 10 typically includes an excitation source 12 and a detector 16. For example, the source 12 may be a Quartz tungsten-halogen source lamp with a 5 position excitation filter wheel by Agilent Technologies (Santa Clara, Calif.). The detector 16 may be e.g., a single scanning photomultiplier tube (PMT) with a 5 position filter wheel by Agilent Technologies (Santa Clara, Calif.). In embodiments, components of the excitation source 12 and detector 16 may be combined. For example, a filter wheel with 5 user-selectable filters for excitation and detection may be used. U.S. patent application publication number 2006/0289786 further provides an example of light sources and detectors for use in QPCR and is herein incorporated by reference in its entirety.

The QPCR system 10 may also include a thermal system 14, e.g., for processing nucleic acid samples. For example in preferred embodiments, the thermal system 14 comprises a solid state, Peltier-based 96-well block thermal system, that can accept, for example, standard (96-tube) plates, 8-tube strips, or single 200 ul tubes. In embodiments, the QPCR system 10 may comprise an Mx3000P™, Mx3005P™, or Mx4000™, etc. QPCR system by Agilent Technologies (Santa Clara, Calif.) and QPCR software such as MxPro™ by Agilent Technologies (Santa Clara, Calif.).

The system 10 may further comprise at least one processor 18 such as an Intel® Celeron® 1.7 GHz (or higher) with e.g., a 20 GB hard drive. The processor 18 may also comprise at least 256 MB RAM and 24X CD-RW, an integrated network card and sound card. The system 10 may also include a display 22, e.g., a 14.1 XGA display, and user input device 20 (such as keyboard, mouse, pointer, or the like). In addition, system 10 components may be in communication with one another using any conventional means, such as wires, buses, wireless connections, or the like. In yet another embodiment of the invention, a storage medium 24 is provided for storing computer programs, files, data, etc. The storage medium 24 may be any of the known media for long-term or short-term storage of computer information. In some embodiments, the storage medium is a portable storage medium, which can be inserted and removed from the processor 18. Examples of storage media include RAM, ROM, optical disk, magneto-optical disk, CD-ROM, CD-R, CD-RW, magnetic tape, memory sticks, nonvolatile memory card, DVD, or the like. In addition, the processor 18 may be equipped with software such as Microsoft® Windows® XP Professional, Microsoft® Office XP Professional, Office 2003 SBE, etc.

Additional QPCR components may be provided separately or integrated with the excitation source, detector, thermal system, and/or processor. These may include optics (such as lenses, filters, and the like) to direct the excitation energy to the samples and direct energy (e.g., fluorescence) emitted from the sample to the detector. For example, the optical system may use scanning fiber optics with a photomultiplier tube (PMT). In addition, spectral analyzers, charge coupled device (CCD) detectors, and the like may also be used for detection and analysis. It is to be understood that the components shown are merely illustrative, and that different combinations of components and parts may be used as necessary for performing PCR experiments according to the disclosed principles of the present invention. For example, those skilled in the art will appreciate that other instruments or methods used in connection with nucleic acid amplification are within the spirit and scope of the present invention.

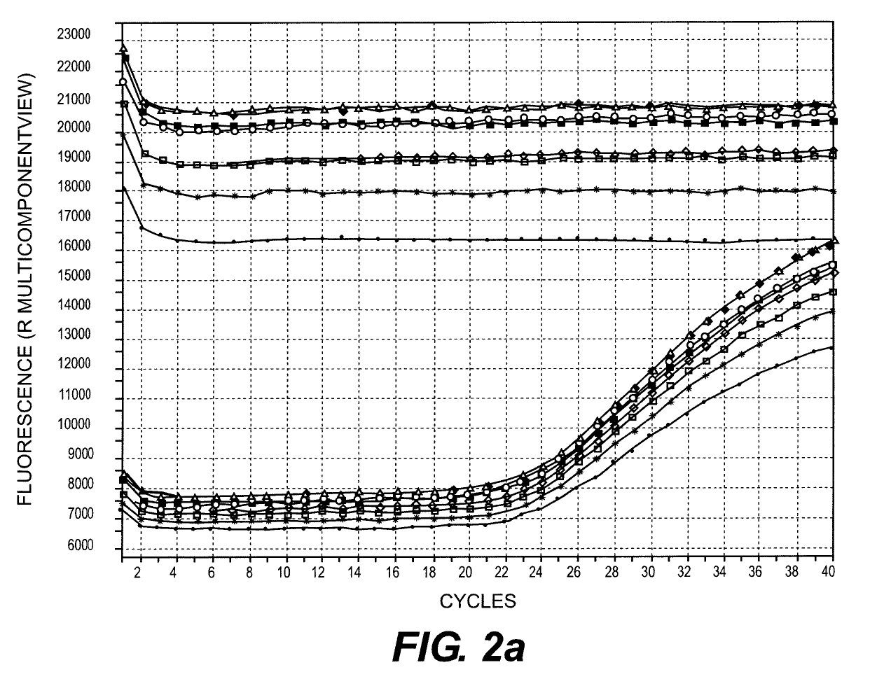

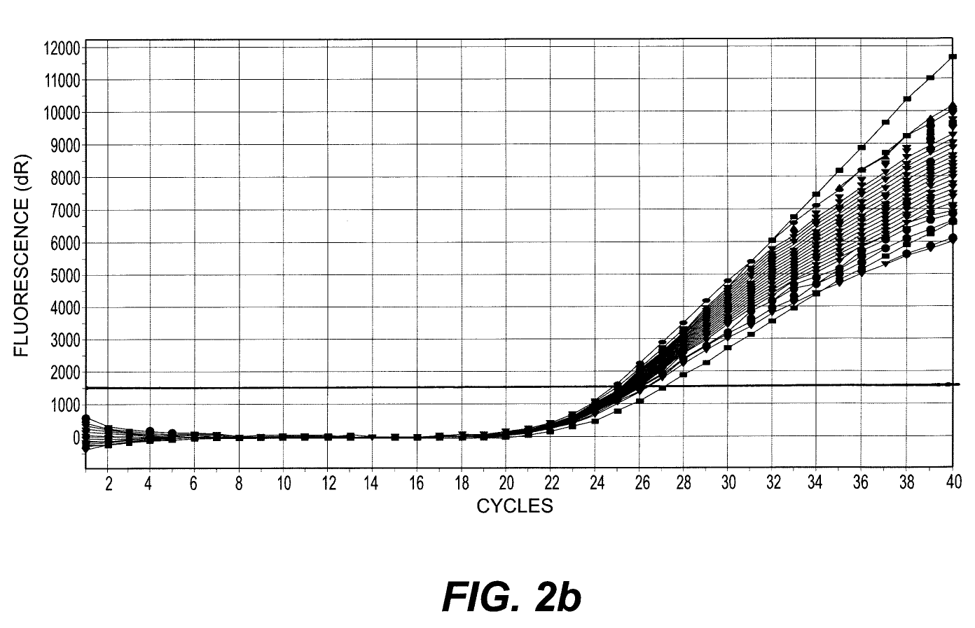

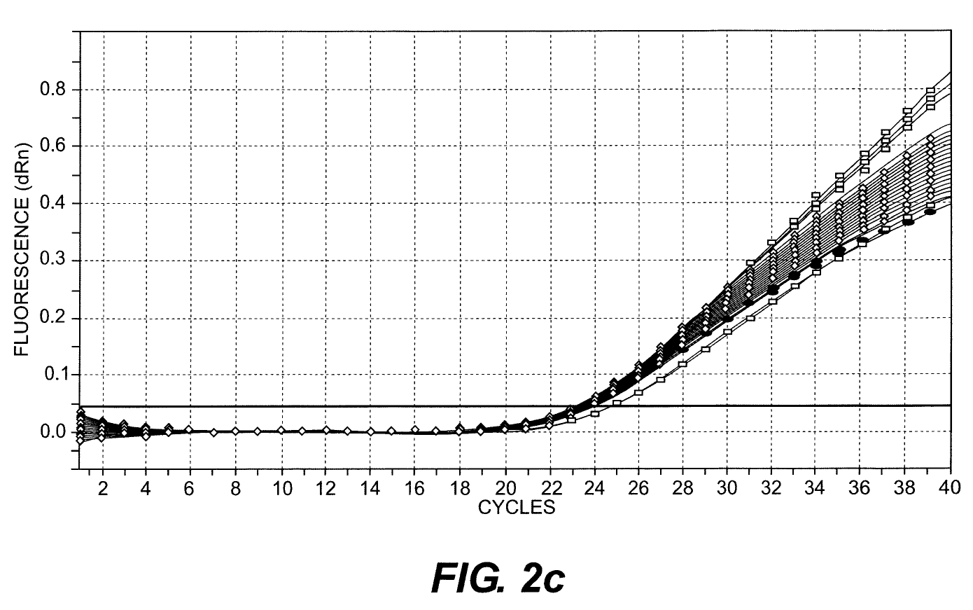

FIG. 2a demonstrates an amplification plot of raw fluorescent intensity signals (y-axis) of a normalizer and probe with respect to cycle number (x-axis). The bottom portion of the plot shows fluorescent intensity of probe signals for each cycle. Each separate line represents signals from a different sample. Signal level differences from the different samples are reflected by differences in intensity between the separate lines. The normalizer signals are shown in the upper portion of the plot and remain relatively constant over many cycles. Again, each separate line represents signals from a different sample. It is observed that while the normalizer signals may experience a small amount of drift, all of the normalizer signals drift similarly. Fluorescent dyes commonly used for QPCR include SYBR™ Green I, TaqMan™, RiboGreen™, PicoGreen™, Alexa350, DABCYL, Cy2, Fluorescein (FAM), Tetrachlorofluorescein, Yakima Yellow, Cy3, TAMRA, ROX, Texas Red™, Malachite Green, Cy5, Cy7, etc. In addition, those skilled in the art will appreciate that other dyes known in the art can be used and are within the spirit and scope of the present invention.

FIG. 2b demonstrates a baseline subtracted (dR) QPCR amplification plot. Baseline subtraction generally helps to reduce the effect of sample-to-sample offset variability (e.g., due to reflection differences between samples) and linear signal drift (e.g., due to lamp degradation). FIG. 2c demonstrates a normalized (dRn) baseline subtracted PCR amplification plot. As can be seen by a reduced spread in intensity values, the normalization helps to account for signal level differences from different samples (e.g., due to sample-to-sample pipetting variability, transmission variability, or gain variability).

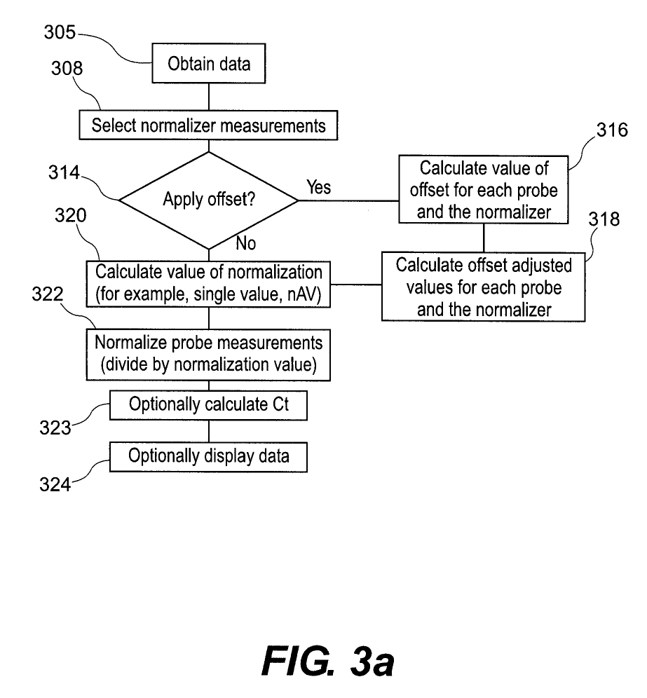

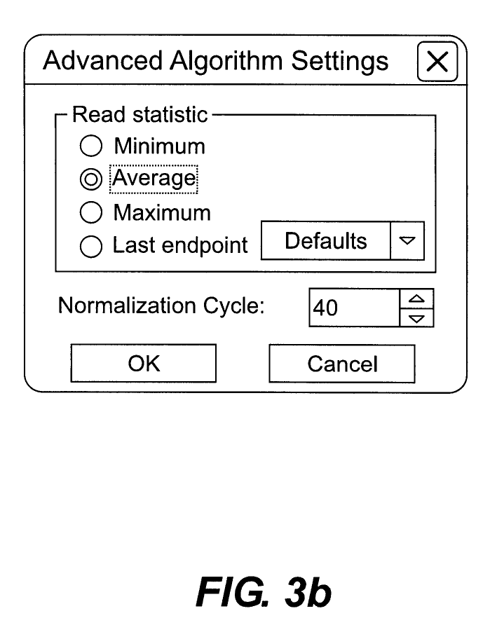

FIG. 3a illustrates exemplary method steps according to a preferred embodiment of the present invention as shown generally at 300. At step 308, a user may select which normalizer measurements to use for normalizing the probe. For example, the user may select the last measurement (R_Last) to be used. In another example, options such as selecting an average normalizer (nAV) measurement may be provided. In some cases, no selection by the user is interpreted as a default selection or input by the user (where the default may be set to R_Last, etc.).

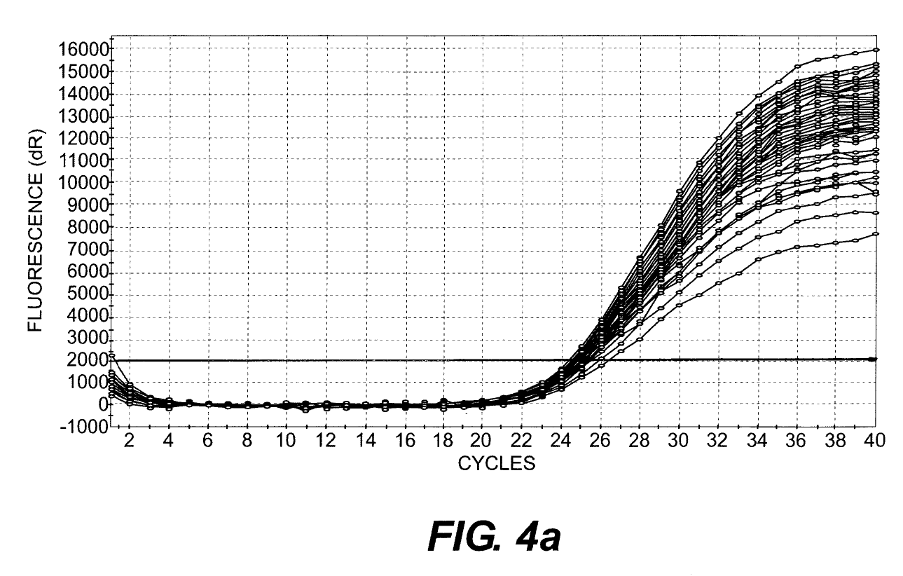

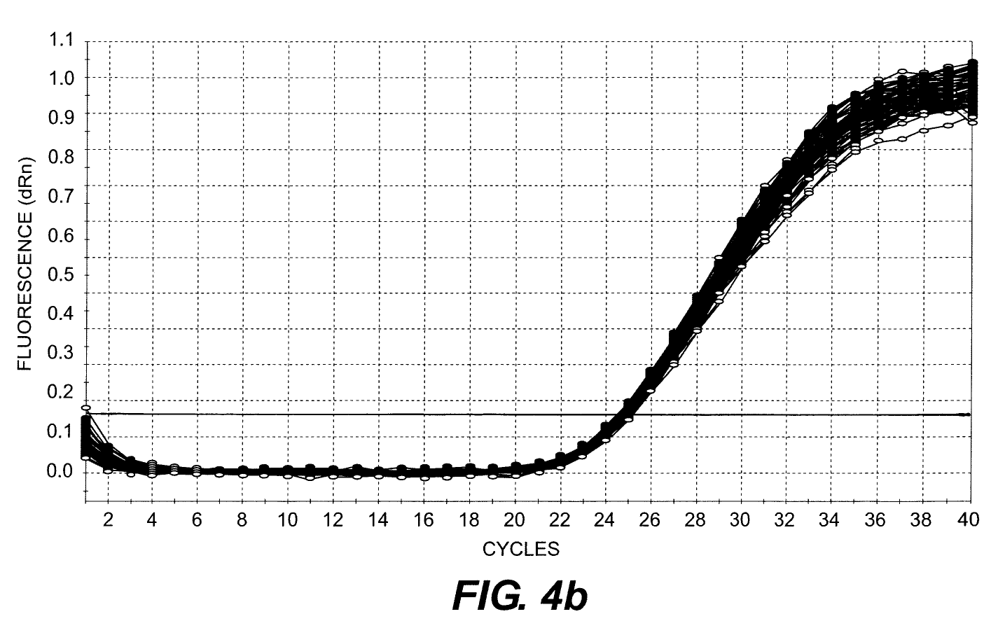

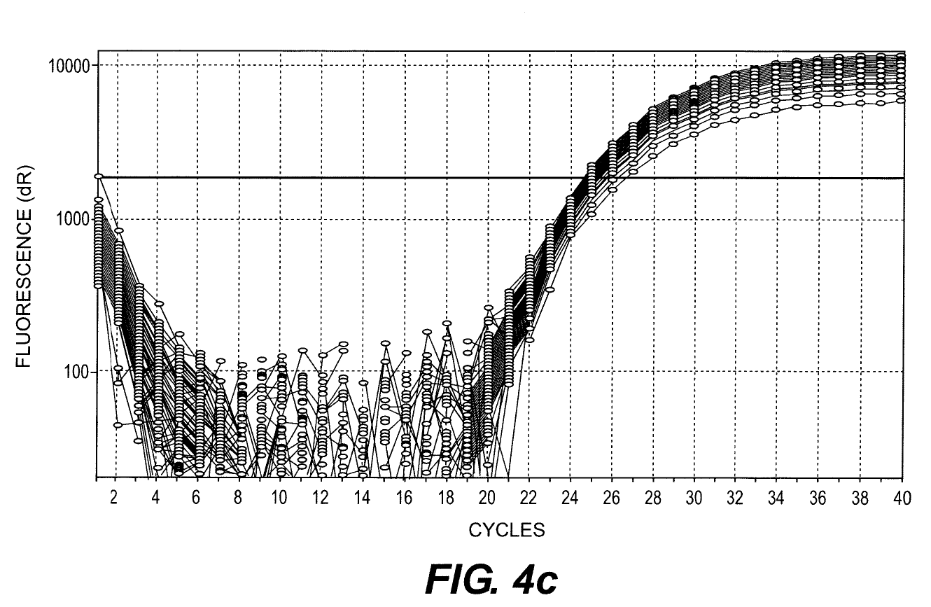

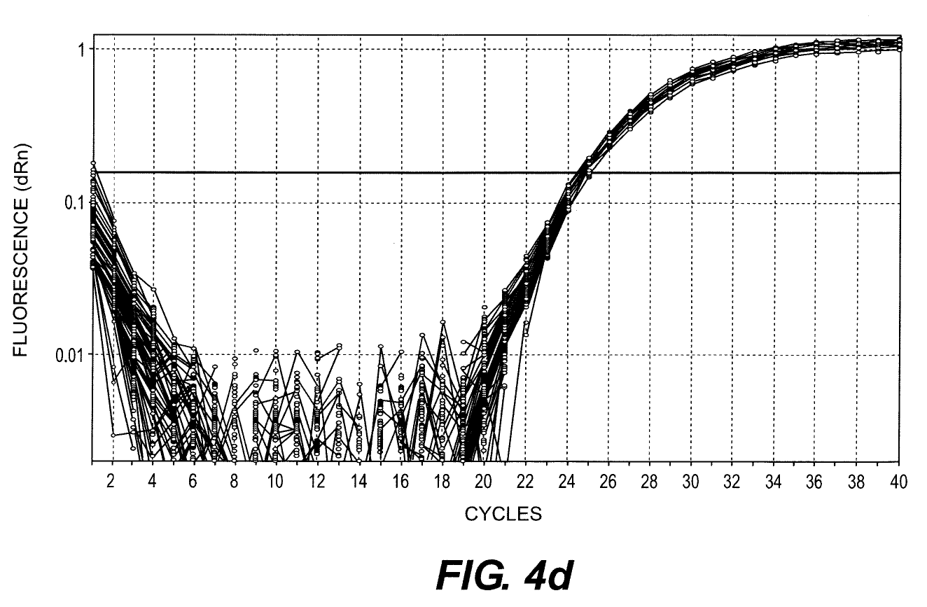

User selections may be made using a graphical user interface (herein termed “MxGUI”). The MxGUI (output from which appear, for example, in FIGS. 2 and 4) preferably provides drop-down menus, selection buttons, or the like, for allowing user-selectable parameters to be set for QPCR procedures. Exemplary user-selection buttons are shown in FIG. 3b. For example, the user may select which cycle's measurement of the normalizer (such as R_Last) to use for normalization or an average measurement (nAVG). In addition, other advanced algorithms may be available for analysis such as those disclosed in U.S. patent application publication number 2005/0255483, herein incorporated by reference in its entirety.

Measurements of one or more probe are obtained using conventional QPCR measurement techniques at step 305. For example, measurements of a single probe or multiple probes (as used in multiplexed PCR experiments) may be obtained. After collecting data and possible user inputs, the algorithm may, at 314, make a decision whether to apply an offset to the probe and/or normalizer data. If the decision is yes, the algorithm calculates the offset at 316 and applies the offset at 318 to all of the appropriate measurements and then proceeds to calculate the normalization values at step 320. If the decision to apply the offset is no, the algorithm proceeds directly to the calculate normalization values at step 320.

The probe measurements may be normalized at 322 by dividing the raw intensity data by the measured normalizer intensity. In preferred embodiments, all of the probe measurements of the sample are normalized by a single normalization measurement from the sample. In embodiments, all of the probe measurements of the sample are normalized by a calculated average of normalizer measurements from the sample. For example, the calculated average may be the mean of two or more normalizer measurements. Because all the probe measurements from a sample are normalized by the same normalization measurement from that sample, the cycle times may effectively be reduced. In addition, baseline subtraction can further be performed either before or after normalization. It will be appreciated by those skilled in the art that the discussed method steps need not necessarily be in the same order shown, and that additional QPCR method steps may be within the spirit and scope of the present invention. For example, in some cases, the probe measurements may be normalized during data collection rather than after the completion of an assay.

According to an aspect of the present invention, the steps shown in FIG. 3a comprise a “single-point” normalization algorithm. In embodiments, the single-point normalization algorithm uses measurements of the normalizer signal at no more than one cycle to normalize all the cycles. Every cycle measurement of a probe may be divided by the same measurement of the normalizer. In further embodiments, the user is given a choice of which cycle to use, with a default to the last cycle (since later cycles may give more stable measurements than earlier cycles). In other embodiments the one measurement of the normalizer may comprise an average of a number of measurements.

After the algorithm normalizes the measurements it may use those measurement to calculate Ct values at 323. It may also display the results from any or all steps of the calculations at 324.

The above method steps may further be implemented in software using conventional software languages such as (C++, Visual Basic, Java, etc.). Additionally or alternatively, the software may be implemented e.g., in the form of an advanced algorithm and/or add-on to existing QPCR software such as MxPro™ by Agilent Technologies (Santa Clara, Calif.). It will further be appreciated by those skilled in the art that other hardware components and/or configurations for performing QPCR may be within the spirit and scope of the present invention.

In addition, the software can be used to analyze R, dR, or dRn. R refers to the raw fluorescent reading, dR refers to the baseline subtracted fluorescent reading, and dRn refers to the baseline subtracted fluorescent reading normalized with the reference dye. Typically, dRn is analyzed when a reference dye is used in the experiment, and dR is analyzed when no reference dye is used. When analyzing fluorescent probes, primers, etc. optimization usually involves observing the lowest Ct values for the primer combinations. Preferably, the software of the present invention determines the Ct value for each sample based upon the parameters selected by the user or by using the default settings.

### EXAMPLES

The invention will now be further described with reference to certain exemplary embodiments and features. It is to be understood that the invention is not limited by the particular examples provided.

### Example 1

### Use of the Invention to Normalize PCR Reactions

Aspects by which the normalization methods of the present invention improve the usefulness of calculations of Ct values and decrease signal variability are addressed with respect to FIGS. 4a-d. FIGS. 4a and c depict dR data (4a uses a linear plot, 4c uses a semi-log plot), and FIGS. 4b and d depict dRn data (4b uses a linear plot, 4d uses a semi-log plot), all from the same experiment. The scales are set based on the range of the data. The dRn plots show normalized signal fluorescence intensities. The Ct values for all plots correspond to the cycle number at which the signal intensities cross a specified threshold.

After normalization of the dR data, the dRn plots shown in FIGS. 4b and d exhibit a tighter range of Ct values. This effect results from the way that the normalization compensates for different signal levels from different samples. If a sample has a low signal, it will appear to have a higher Ct value (corresponding to decreased sensitivity) than a sample with a stronger signal. Because the normalization compensates for signal differences that can mask amplification differences, the amplification differences between samples play a more significant role. In other words, normalization reduces the signal level variability between samples that is unrelated to the starting concentrations of the samples and thereby allows more accurate and precise Ct value calculations. The spread of the final dRn and dR signals further shows that the normalization reduced the spread (and thereby variability) of the signals. In this example, the standard deviation of the Ct values divided by the average Ct value for dRn is 0.72%; that of dR is 2.03%.

### Example 2

### Comparison of Embodiments of the Invention

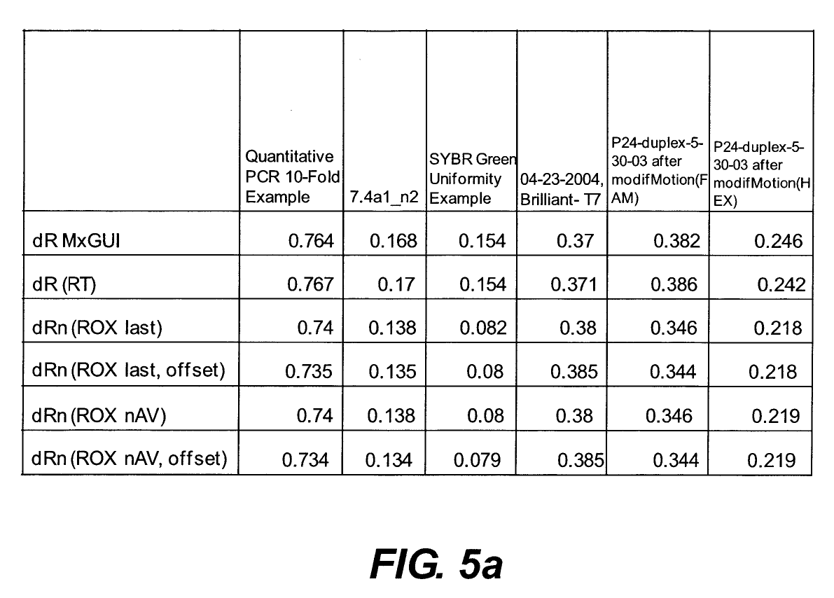

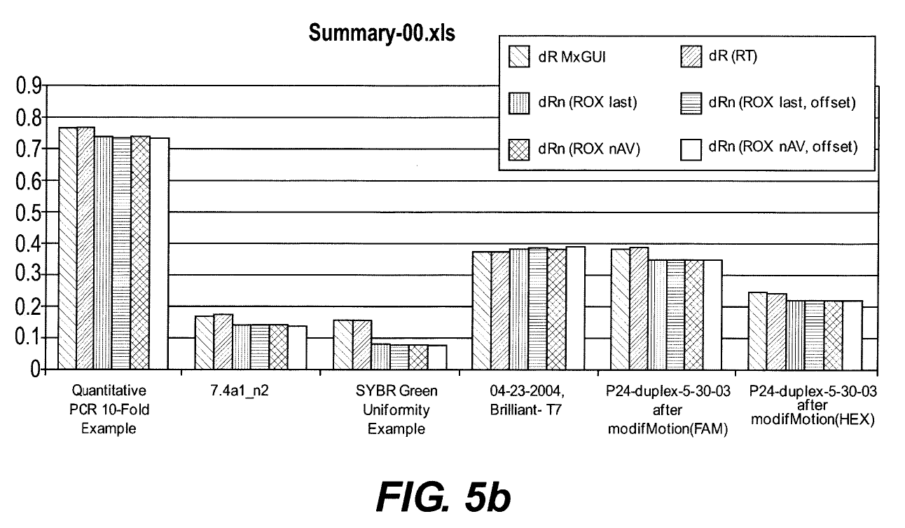

An experiment was performed to compare the following three variations of the “single-point” algorithm: 1) dividing every measurement of the probe by the last measurement of the normalizer; 2) dividing every measurement of the probe by the average of the last four measurements of the normalizer; and 3) subtracting constant values from the probe and/or normalizer signals before performing the normalization. The results are shown in FIGS. 5a and 5b, which are discussed in more detail below.

The comparisons used measurements from various QPCR assays. The Ct values were calculated in all cases using a noise-based threshold (10 sigma). The root-mean-square (rms) values of the sigmas of the Ct values were compared from each replicate of a run across the different algorithms.

FIG. 5a summarizes in tabular form the spread of the Ct values determined from several experiments using the different normalization algorithms (columns organize results by assay, rows by algorithm). The graph in FIG. 5b illustrates the data of FIG. 5a in graphical form, plotting the spread of Ct values for different normalization algorithms. As shown in this Figure, the results from the MxQPCR software are indicated by diagonal fill lines. These values were used as a check that the algorithms were implemented correctly. The un-normalized dR results are the first two bars for each assay example. The last four bars for each experiment are the single-point (sp) normalized results (last point, last point offset, average of the last 4 points, average of last 4 points offset).

For these experiments, the two single-point algorithms gave nearly the same results. Including the offset in the algorithm improved the results slightly for most of the experiments (although good offset values were not present for all of the instruments).

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. All patents, patent applications and published references cited herein are incorporated by reference in their entirety. Furthermore, it is intended that the specification and examples be considered as exemplary only, with a true scope and spirit of the invention being indicated by the following claims.

1. A method for normalizing data in a nucleic acid amplification procedure performed on a sample, said method comprising:
a) obtaining raw intensity measurements of one or more probes over multiple cycles of the procedure;
b) obtaining one or more normalizer intensity measurements from the sample;
c) calculating a single value from the normalizer intensity measurements of the sample; and
d) using the single calculated normalizer value to normalize all the probe raw intensity measurements of the sample.

2. The method of claim 1, further comprising receiving input from a user regarding which normalizer measurements to use for normalization.

3. The method of claim 1, wherein only one normalizer measurement is obtained in step b).

4. The method of claim 3, further comprising obtaining the normalizer measurement before performing a cycle of the procedure.

5. The method of claim 3, further comprising obtaining the normalizer measurement after completing all cycles of the procedure.

6. The method of claim 1, further comprising:
obtaining two or more normalizer measurements in step b), calculating the average value of the normalizer measurements, and using the average value in step c).

7. The method of claim 6, further comprising obtaining two or more normalizer measurements before performing a cycle of the procedure.

8. The method of claim 6, further comprising obtaining two or more normalizer measurements after completion of all cycles of the procedure.

9. The method of claim 1, wherein step c) includes dividing each probe measurement by the calculated normalizer value.

10. The method of claim 1, wherein step c) includes subtracting an offset value from the measurements before dividing each probe measurement by the obtained normalizer measurements.

11. The method of claim 1, further comprising displaying the normalized data on a display.

12. The method of claim 1, further comprising using steps a)-c) in a calculation to produce a Ct value for one or more probe.

13. A system for normalizing data in nucleic acid amplification procedures, said system comprising:
a) means for obtaining from a sample raw intensity measurements of one or more probes over multiple cycles of the procedure;
b) means for obtaining one or more normalizer intensity measurements from the sample;
c) means for calculating a single value from the normalizer intensity measurements of the sample; and
d) means for using the single calculated normalization value in normalizing the probe measurements of a sample.

14. The system of claim 13, wherein the means for obtaining in step b) is configured to obtain one normalizer measurement.

15. The system of claim 13, wherein the means for obtaining in step b) is configured to obtain an average of two or more normalizer measurements.

16. The system of claim 13, wherein the means for normalizing in step d) is configured to divide the probe measurements by the normalizer measurements.

17. The system of claim 13, further comprising a user input means and a display means.

18. The system of claim 13, wherein the system is configured to receive input from a user regarding which normalizer measurements are to be obtained.

19. The system of claim 13, wherein the system is configured to receive input from a user regarding which normalizer measurements are to be used.

20. A computer program for normalizing data in a nucleic acid amplification procedure, said program comprising instructions for:
a) receiving input from a user indicating which normalizer intensity measurements to use in calculations during the procedure;
b) obtaining from a sample raw intensity measurements of one or more probes over multiple cycles;
c) obtaining one or more normalizer measurements;
d) calculating a single value from the normalizer intensity measurements of the sample;
e) normalizing the probe measurements with respect to the single calculated normalizer value; and
f) displaying the normalized probe measurements on a display.

21. The computer program of claim 20, wherein only one measurement of the normalizer is used in step e).

22. The computer program of claim 20, wherein only last normalizer measurement is obtained in step c).

23. The computer program of claim 20, where an average of two or more normalizer measurements obtained over no more than one cycle is used in step e).

24. The computer program of claim 20, wherein steps b)-e) are part of a calculation that produces a Ct value for one or more probe.

25. A computer program product residing on a computer readable medium, said program comprising instructions for normalizing data in a nucleic acid amplification procedure, wherein the instructions include:
a) receiving input from a user indicating which normalizer intensity measurements to use during the procedure;
b) obtaining from a sample raw intensity measurements of one or more probes over multiple cycles of the procedure;
c) obtaining one or more normalizer intensity measurements;
d) calculating a single value from the normalizer intensity measurements;
e) normalizing the probe measurements with respect to the single calculated normalizer value; and
f) outputting the normalized probe measurements to a display.
